(19) United States
(12) Patent Application Publication
(10) Pub. No.: US 2026/0033903 A1
(43) Pub. Date: Feb. 5, 2026

(54) INPUT-RECEIVING COMPONENT FOR ROBOTIC MICROSURGICAL PROCEDURES

(71) Applicant: FORSIGHT ROBOTICS LTD., Yokneam Illit (IL)

(72) Inventors: Yoav GOLAN, Tel Aviv (IL); Tal KORMAN, Tel Aviv (IL); Ori BEN ZEEV, Ramat Hasharon (IL)

(21) Appl. No.: 19/354,124

(22) Filed: Oct. 9, 2025

Related U.S. Application Data
(63) Continuation of application No. PCT/IB2024/054525, filed on May 9, 2024.
(60) Provisional application No. 63/465,038, filed on May 9, 2023.

(51) Int. Cl. A61B 34/37; A61B 17/00; A61B 34/00; A61B 34/30; A61F 9/007

(52) U.S. Cl. CPC A61B 34/37; A61B 34/72; A61B 34/74; A61F 9/007; A61B 2017/00057; A61B 2034/302

(57) ABSTRACT

Apparatus and methods are described including a control-component tool that includes a handle and an input-receiving component disposed on the handle. The input-receiving component includes one or more light sources disposed on a first radial wall, one or more light detectors disposed on a second radial wall that faces the first radial wall, and a deformable sleeve extending axially between the first and second radial walls and configured to attenuate light that is directed from the one or more light sources toward the one or more light detectors, in response to being pressed. A computer processor receives a signal from the one or more light detectors that is indicative of the light attenuation and controls a surgical tool in response thereto. Other applications are also described.

### CROSS-REFERENCES TO RELATED APPLICATIONS

The present application is a continuation of PCT Application No. PCT/IB2024/054525 to Golan et al., filed May 9, 2024 (published as WO 24/231879), entitled “Input-receiving component for robotic microsurgical procedures,” which claims priority from U.S. Provisional Patent Application No. 63/465,038 to Golan et al., filed May 9, 2023, entitled “Input-receiving component for robotic microsurgical procedures,” which is incorporated herein by reference.

### FIELD OF EMBODIMENTS OF THE INVENTION

Some applications of the present invention generally relate to medical apparatus and methods. Specifically, some applications of the present invention relate to apparatus and methods for performing microsurgical procedures in a robotic manner.

### BACKGROUND

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and their face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye via the corneal incision(s), in order to further anesthetize the eye. Following this step, the pupil is dilated, and a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed using one or more tools inserted via the corneal incision(s). Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incision(s), in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. When the phacoemulsification is complete, the remaining lens cortex (i.e., the outer layer of the lens) and viscoelastic material is aspirated from the capsule. During the phacoemulsification and the aspiration, aspirated fluids are typically replaced with irrigation of a balanced salt solution, in order to maintain fluid pressure in the anterior chamber.

In some cases, if deemed to be necessary, the capsule is polished. Subsequently, the intraocular lens (IOL) is inserted into the capsule. The IOL is typically foldable and is inserted in a folded configuration, before unfolding inside the capsule. If necessary, the one or more of the incisions are sealed by elevating the pressure inside the bulbus oculi (i.e., the globe of the eye), causing the internal tissue to be pressed against the external tissue of the incisions, such as to force closed the incisions.

### SUMMARY

In accordance with some applications of the present invention, a robotic system is provided that is configured for use in a microsurgical procedure, such as ophthalmic surgery. Typically, when used for ophthalmic surgery, the robotic system includes one or more robotic units (which are configured to hold surgical tools), in addition to an imaging system, one or more displays and a control-component unit (e.g., a control-component unit that includes a pair of control components), via which one or more operators (e.g., healthcare professionals, such as a physician and/or a nurse) are able to control the robotic units. Typically, the robotic system includes one or more computer processors, via which components of the system and operator(s) operatively interact with each other.

Typically, the control-component unit includes one or more (e.g., a pair of) control components that are configured to correspond to respective robotic units of the robotic system. For example, the system may include first and second robotic units, and the control-component unit may include first and second control components, as shown in FIG. 1A for example. Typically, each of the control components includes an arm that includes a plurality of links that are coupled to each other via joints. For some applications, the control components include respective control-component tools (which are typically configured to replicate the surgical tools). Typically, the computer processor determines the XYZ location and orientation of the tip of the control-component tool, and drives the robotic unit such that the tip of the actual tool that is being used to perform the procedure (i.e., the surgical tool) tracks the movements of the tip of the control-component tool and such that changes in the orientation of surgical tool track changes in the orientation of the control-component tool.

Typically, movement of the robotic units (and/or control of other aspects of the robotic system) is at least partially controlled by the one or more operators. For example, the operator may receive images of the patient's eye and the robotic units and/or tools disposed therein, via the display. Typically, such images are acquired by the imaging system. For some applications, the imaging system includes a stereoscopic imaging device and the display is a stereoscopic display. Based on the received images, the operator typically performs steps of the procedure. For some applications, the operator provides commands to the robotic units via the control-component unit. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), forceps (e.g., opening and closing of forceps), an intraocular-lens-manipulator tool (e.g., such that the tool manipulates the intraocular lens inside the eye for precise positioning of an intraocular lens within the eye), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, orientation, and/or XYZ positioning of the imaging system). For some applications, the control-component tool (and/or a different portion of the control-component unit) includes one or more input-receiving components that are configured to receive such inputs from the operator.

For some applications, the input-receiving component is configured to be rotationally agnostic, such that the input that is detected is the same regardless of the roll orientation of the control-component tool relative to the operator's hand. In some cases, this is desirable, since the control-component tool typically undergoes roll angular rotation relative to the operator's hand during use. For some applications, the one or more input-receiving components are disposed at discrete circumferential positions with respect to the control-component tool, such that the input-receiving components can only be pressed at certain circumferential positions. In some cases this is desirable, in order for the input-receiving components to more closely resemble those of conventional surgical tools such as forceps.

For some applications, the input-receiving component includes a deformable sleeve, one or more light sources, and one or more light detectors. For some applications, the deformable sleeve comprises an elastomeric material, such as silicone. For some applications, the handle of the control-component tool defines a first radial wall (i.e., a wall, a surface of which is disposed along the radial direction of the handle) and the one or more light sources include a plurality of LEDS positioned circumferentially upon the first radial wall such that the LEDs direct light parallel to the axis of the handle. For some applications, the handle defines a second radial wall that faces the first radial wall, and the one or more light detectors include one more ambient light detectors that are disposed circumferentially upon the second radial wall such that the light detectors face the LEDs. Typically, the deformable sleeve extends axially between the first and second radial walls.

For some applications, the control-component unit is configured such that the operator provides an input to the robotic system by pressing the deformable sleeve. Typically, the pressure on the deformable sleeve causes the sleeve to deform, and at least partially attenuate light that is directed from the light sources toward the light detectors. The computer processor typically receives a signal that is indicative of the light attenuation from the light detectors and controls the robotic system and/or the imaging system in response to the detected light attenuation. For some applications, based upon the level and/or type of light attenuation, the computer processor detects the extent to which the sleeve has been deformed (which is indicative of the amount of pressure that has been applied by the operator) and controls the robotic system and/or the imaging system in response thereto. For example, the amount of force with which forceps are closed by the robotic system may be controlled in response to the amount of pressure that has been applied by the operator. For some applications, the input-receiving component is configured to receive an input via pressure that is applied to the deformable sleeve by a single finger or thumb of the operator. Alternatively or additionally, the input-receiving component is configured to receive an input via pressure that is applied to the deformable sleeve by two fingers (or a finger and a thumb), for example in a squeezing action.

It is noted that the input-receiving component as described above typically does not rely upon rigid mechanical motion of any components in order for the computer processor to detect an input by the operator. Thus, the input-receiving component is not susceptible to mechanical breakdown that is typical of components that do rely upon such rigid mechanical motion.

For some applications, the input-receiving component includes one or more magnetic structures (e.g., magnetic leaf springs arranged parallel with the axis of the handle). Typically, the magnetic structures are covered with a flexible sleeve, such as an elastomeric sleeve (e.g., a silicone sleeve). For some applications, one or more magnetic materials are embedded within the sleeve. For some applications, the sleeve itself is magnetic. For example, the sleeve may include magnetic silicone. Typically, one or more magnetometers (e.g., Hall sensors) are disposed within the handle and are configured to measure magnetic flux that is generated by the magnetic structures in response to pressure being applied to the magnetic structures by the operator.

There is therefore provided, in accordance with some applications of the present invention, apparatus for performing a procedure on a patient using a surgical tool, the apparatus including:
a robotic unit configured to control the surgical tool; and
a control-component tool that is configured to be held an operator, the control-component tool including:
   a handle; and
   an input-receiving component disposed on the handle that includes:
      a first radial wall with one or more light sources disposed on the first radial wall;
      a second radial wall that faces the first radial wall, with one or more light detectors disposed on the second radial wall;
      and a deformable sleeve extending axially between the first and second radial walls and configured to attenuate light that is directed from the one or more light sources toward the one or more light detectors, in response to being pressed; and
a computer processor configured to:
   receive a signal from the one or more light detectors that is indicative of the light attenuation; and
   control the surgical tool in response thereto.

In some applications, the input-receiving component does not rely upon rigid mechanical motion of any components in order for the computer processor to detect an input to the input-receiving component.

In some applications, the input-receiving component is configured to receive an input via pressure that is applied to the deformable sleeve by a single finger or thumb of an operator.

In some applications, the input-receiving component is configured to receive an input via pressure that is applied to the deformable sleeve by two fingers.

In some applications, the light sources and light detectors are distributed non-uniformly around a circumference of the handle and the attenuation of the light in response to the deformable sleeve being pressed only occurs at discrete locations around the circumference of handle.

In some applications, the apparatus is configured for performing an ophthalmic procedure on an eye of a patient using one or more surgical tools that have tips and the robotic unit is configured to move the one or more surgical tools within the patient's eye.

In some applications, the computer processor is configured to:
determine movement of the location and orientation of the tip of the control-component tool based upon data received from the one or more location sensors; and
move the tip of the surgical tool within the patient's eye in a manner that corresponds with the movement of the location and orientation of the tip of the control-component tool.

In some applications, the input-receiving component is configured to be rotationally agnostic, such that the attenuation of the light in response to the deformable sleeve being pressed is the same regardless of a roll orientation of the control-component tool relative to an operator's hand.

In some applications, the light sources and light detectors are distributed uniformly around a circumference of handle.

There is further provided, in accordance with some applications of the present invention, apparatus for performing a procedure on a patient using a surgical tool, the apparatus including:
a robotic unit configured to control the surgical tool; and
a control-component tool that is configured to be held an operator, the control-component tool including:
   a handle; and
   an input-receiving component disposed on the handle that includes:
      one or more magnetic leaf springs; and
      a magnetometer; and
a computer processor configured to:
   receive a signal from the magnetometer that is indicative of the one or more magnetic leaf springs being pressed; and
   control the surgical tool in response thereto.

In some applications, the magnetic leaf springs are covered with a flexible sleeve.

In some applications, the input-receiving component is configured to receive an input via pressure that is applied to the magnetic leaf springs by a single finger or thumb of an operator.

In some applications, the input-receiving component is configured to receive an input via pressure that is applied to the magnetic leaf springs by two fingers.

In some applications, the magnetic leaf springs are distributed non-uniformly around a circumference of the handle.

In some applications, the apparatus is configured for performing an ophthalmic procedure on an eye of a patient using one or more surgical tools that have tips and the robotic unit is configured to move the one or more surgical tools within the patient's eye.

In some applications, the computer processor is configured to:
determine movement of the location and orientation of the tip of the control-component tool based upon data received from the one or more location sensors; and
move the tip of the surgical tool within the patient's eye in a manner that corresponds with the movement of the location and orientation of the tip of the control-component tool.

In some applications, the input-receiving component is configured to be rotationally agnostic, such that magnetic flux that is generated by pressing the magnetic leaf springs is the same regardless of a roll orientation of the control-component tool relative to an operator's hand.

In some applications, the magnetic leaf springs are distributed uniformly around a circumference of handle.

There is further provided, in accordance with some applications of the present invention, apparatus for performing a procedure on a patient using a surgical tool, the apparatus including:
a robotic unit configured to control the surgical tool; and
a control-component tool that is configured to be held an operator, the control-component tool including:
   a handle;
   a first magnet disposed on a first side of a longitudinal axis of the handle and a second magnet disposed opposite the first magnet on a second side of the longitudinal axis of the handle, with a pole of each of the first and second magnets that is disposed closer to the longitudinal axis being the same as each other; and
   a magnetometer that is disposed within the handle along a centerline of the first and second magnets along a radial direction of the handle but offset from centerlines of the first and second magnets along an axial direction of the handle;
a computer processor configured to:
   receive a signal from the magnetometer that is indicative of magnetic flux that is generated by the magnetic structures being pressed; and
   control the surgical tool in response thereto.

In some applications, the computer processor is configured to distinguish between the only the first magnet being pressed, only the second magnet being pressed, and both magnets being pressed based on the signal from the magnetometer.

In some applications, the computer processor is configured to determines a total pressure with which the magnets have been pressed based on the signal from the magnetometer.

In some applications, the first and second magnets include one or more leaf springs.

In some applications, the apparatus is configured for performing an ophthalmic procedure on an eye of a patient using one or more surgical tools that have tips and the robotic unit is configured to move the one or more surgical tools within the patient's eye.

In some applications, the computer processor is configured to:
determine movement of the location and orientation of the tip of the control-component tool based upon data received from the one or more location sensors; and
move the tip of the surgical tool within the patient's eye in a manner that corresponds with the movement of the location and orientation of the tip of the control-component tool.

There is further provided, in accordance with some applications of the present invention, apparatus for performing a procedure on a patient using a surgical tool, the apparatus including:
a robotic unit configured to control the surgical tool; and
a control-component tool that is configured to be held an operator, the control-component tool including:
   a handle; and
   an input-receiving component disposed on the handle that includes:
      a piston barrel defining a neck and conical head;
      one or more spring wires, each of the spring wires having a first portion that protrudes radially from a housing of the handle and a second portion that contacts the conical head of the piston barrel, such that pressure upon the first portion of the spring wire causes the second portion of the spring wire to push the piston barrel axially;
      a magnet disposed within the neck of the piston barrel; and
      a magnetometer configured to detect magnetic flux generated by movement of the magnet; and
a computer processor configured to:
   receive a signal from the magnetometer that is indicative of the one or more spring wires being pressed; and
   control the surgical tool in response thereto.

There is further provided, in accordance with some applications of the present invention, apparatus for performing a procedure on a patient using a surgical tool, the apparatus including:
a robotic unit configured to control the surgical tool; and
a control-component tool that is configured to be held an operator, the control-component tool including:
   a handle; and
   an input-receiving component disposed on the handle that includes:
      a sleeve disposed on the handle;
      one or more ribs protruding radially inwardly from the sleeve;
      a shaft disposed concentrically with the sleeve inside the sleeve; and
      one or more pressure sensors disposed on the shaft and configured to be pressed by the one or more ribs in response to the sleeve being pressed; and
a computer processor configured to:
   receive a signal from the one or more pressure sensors that is indicative of the one sleeve being pressed; and
   control the surgical tool in response thereto.

There is further provided, in accordance with some applications of the present invention, apparatus for performing a procedure on a patient using a surgical tool, the apparatus including:
a robotic unit configured to control the surgical tool; and
a control-component tool that is configured to be held an operator, the control-component tool being configured to undergo roll angular rotation, the control-component tool including:
   a handle including a housing; and
   inner and outer roll-limiting components disposed within the housing, the inner roll-limiting component being disposed within the outer roll-limiting component,
      the inner roll-limiting component having a circular cross-section with a radial protrusion, the outer roll-limiting component having a circular cross-section with an inner radial protrusion and an outer radial protrusion, and the housing of the handle having an inner radial protrusion,
      roll of the inner roll-limiting component being limited by the radial protrusion of the inner roll-limiting component being blocked by the inner radial protrusion of outer roll-limiting component, and
      roll of the outer roll-limiting component being limited by outer radial protrusion of the outer roll-limiting component being blocked by the inner radial protrusion of the inner surface of the housing.

The present invention will be more fully understood from the following detailed description of embodiments thereof, taken together with the drawings, in which:

### BRIEF DESCRIPTION OF THE DRAWINGS

FIGS. 1A and 1B are schematic illustrations of robotic systems that are configured for use in a microsurgical procedure, such as ophthalmic surgery, in accordance with some applications of the present invention;

FIGS. 2A, 2B, 2C, and 2D are schematic illustrations of a control-component unit that includes a control-component tool, in accordance with some applications of the present invention;

FIGS. 2E, 2F, 2G, and 2H are schematic illustrations of a control component and a control-component tool of a control-component unit, in accordance with some alternative applications of the present invention;

FIGS. 3A and 3B are schematic illustrations of a cross-sectional view of a handle of a control-component tool that includes an input-receiving component, in accordance with some applications of the present invention;

FIGS. 4A, 4B and 4C are schematic illustrations of portions of the handle of the control-component tool of FIGS. 3A and 3B, in accordance with some applications of the present invention;

FIGS. 5A, 5B, and 5C are schematic illustrations of portions of a handle of a control-component tool that includes an input-receiving component, in accordance with some alternative applications of the present invention;

FIGS. 6A, 6B, and 6C are schematic illustrations of respective arrangements of magnets and a magnetometer, in accordance with some applications of the present invention;

FIGS. 7A and 7B are graphs indicating changes in magnetic flux that are generated along respective directions using the arrangement shown in FIG. 6A, in accordance with some applications of the present invention;

FIGS. 8A and 8B are schematic illustrations of an oblique view and a cross-sectional view of a handle of a control-component tool that includes an input-receiving component, in accordance with some further alternative applications of the present invention;

FIGS. 9A, 9B, and 9C are schematic illustrations of a handle of a control-component tool that includes an input-receiving component, in accordance with some alternative applications of the present invention;

FIGS. 10A and 10B are schematic illustrations of a handle of a control-component tool that includes an input-receiving component, in accordance with some alternative applications of the present invention; and

FIG. 11 is a schematic illustration of a mechanism for controlling the roll angular rotation of a control-component tool, in accordance with some applications of the present invention.

### DETAILED DESCRIPTION OF EMBODIMENTS

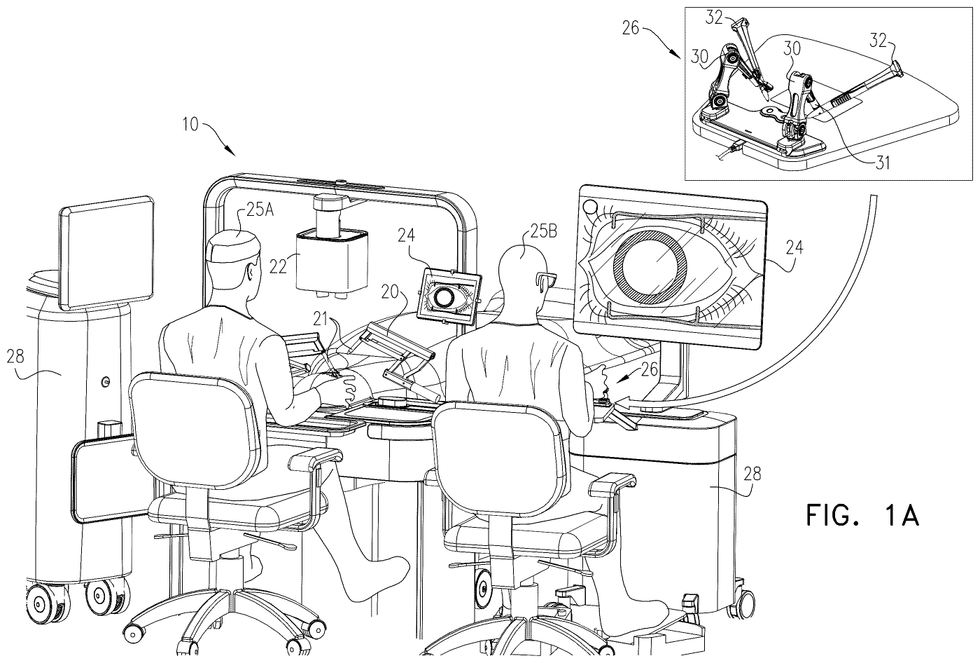

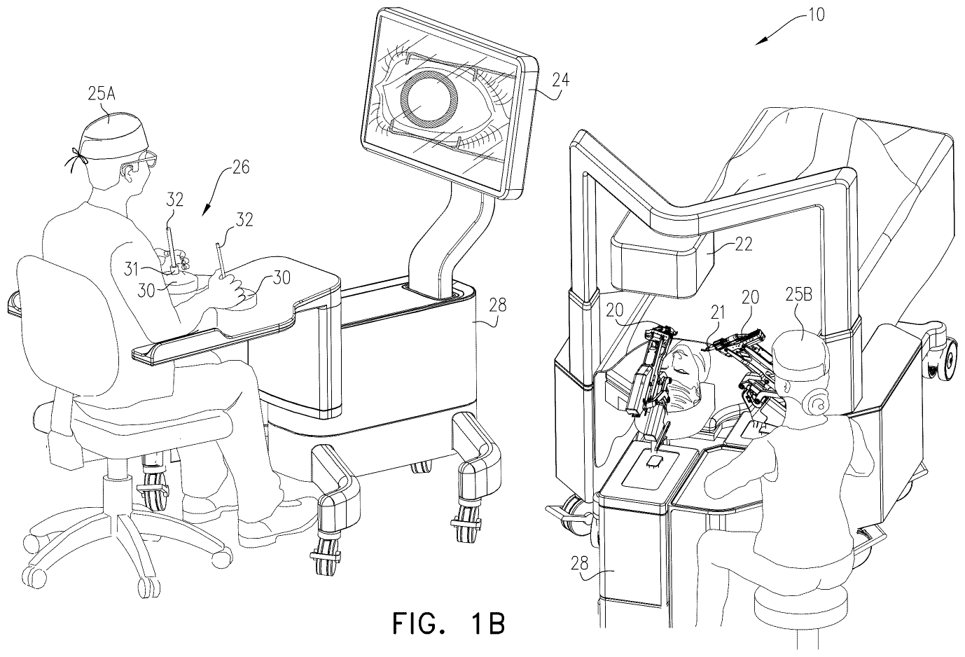

Reference is now made to FIGS. 1A and 1B, which are schematic illustrations of robotic systems 10 that are configured for use in a microsurgical procedure, such as ophthalmic surgery, in accordance with some applications of the present invention. Typically, when used for ophthalmic surgery, robotic system 10 includes one or more robotic units 20 (which are configured to hold tools 21), in addition to an imaging system 22, one or more displays 24 and a control component 26, via which one or more operators 25 (e.g., healthcare professionals, such as a physician and/or a nurse) control robotic units 20. Typically, robotic system 10 includes one or more computer processors 28, via which components of the system and operator(s) 25 operatively interact with each other. The scope of the present application includes mounting one or more robotic units onto a robotic system in any of a variety of different positions with respect to each other.

FIGS. 1A and 1B show different setups of a robotic system 10 that is configured for ophthalmic surgery. As shown, in the configuration shown in FIG. 1A first and second robotic units are disposed at respective lateral positions (i.e., left and right) with respect to the eye that is being operated on, such that tools 21 that are held by the robotic units are disposed at approximately 180 degrees from each other. The configuration shown in FIG. 1B shows a first robotic unit that is placed laterally with respect to the eye and a second robotic unit positioned in a superior position with respect to the eye, such that tools 21 that are held by the robotic units are disposed at approximately 90 degrees from each other. (In the context of ophthalmic procedures, the lateral position shown in FIG. 1B is referred to as the “temporal” position. As such, the terms “lateral” and “temporal” are used interchangeably in the present application.) In some cases (not shown), the first robotic unit is placed laterally with respect to the eye and the second robotic unit positioned in an inferior position with respect to the eye, such that tools 21 that are held by the robotic units are disposed at approximately 90 degrees from each other. In general, the scope of the present disclosure includes using any number of robotic units placed at any number of respective positions in relation to the patient, and the configurations shown in FIGS. 1A and 1B should not be interpreted as limiting the scope of the disclosure in any way.

Typically, movement of the robotic units (and/or control of other aspects of the robotic system) is at least partially controlled by one or more operators 25 (e.g., healthcare professionals, such as a physician and/or a nurse). For example, the operator may receive images of the patient's eye and the robotic units and/or tools disposed therein, via display 24. Typically, such images are acquired by imaging system 22. For some applications, imaging system 22 includes a stereoscopic imaging device and display 24 includes a stereoscopic display. Based on the received images, the operator typically performs steps of the procedure. For some applications, the operator provides commands to the robotic units via control component 26. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), forceps (e.g., opening and closing of forceps), an intraocular-lens-manipulator tool (e.g., such that the tool manipulates an intraocular lens inside the eye for precise positioning of the intraocular lens within the eye), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, orientation, and/or XYZ positioning of the imaging system).

Typically, control component 26 includes one or more control-component units 30 that are configured to correspond to respective robotic units 20 of the robotic system. For example, as shown in FIGS. 1A-1B, the system may include first and second robotic units, and the control component may include first and second control-component units 30. For some applications, the control-component units comprise respective control-component tools 32 therein (in order to replicate the robotic units), as shown in FIGS. 1A-1B. Typically, the computer processor determines the XYZ location and orientation of a tip of the control-component tool 32, and drives the robotic unit such that the tip of surgical tool 21 that is being used to perform the procedure tracks the movements of the tip of the control-component tool.

Typically, the right control-component unit controls movement of the surgical tool that is toward the right of the patient's head when viewing the patient from a superior position (and which would normally be controlled by the physician's right hand), and the left control-component unit controls movement of the surgical tool that is toward the left of the patient's head when viewing the patient from a superior position (and which would normally be controlled by the physician's left hand).

For some applications, while a tool is inserted into the patient's eye via an incision, the computer processor is configured to drive the control-component unit to provide feedback to the operator that is indicative of a location of the entry of the tool into the patient's eye within the incision. For example, as the tool is moved in such a manner that the entry location of the tool into the patient's eye is closer to the edge of the incision, resistance to movement of the control-component arm may be increased, and/or the control-component arm may be vibrated, and/or a different output may be generated. For some applications, the computer processor is configured to apply forces that oppose the operator's attempted movements of control-component tool 32 that would result in the tool being moved beyond the edges of the incision. For some applications, in order to provide the above-described force feedback, the control-component unit includes one or more motors.

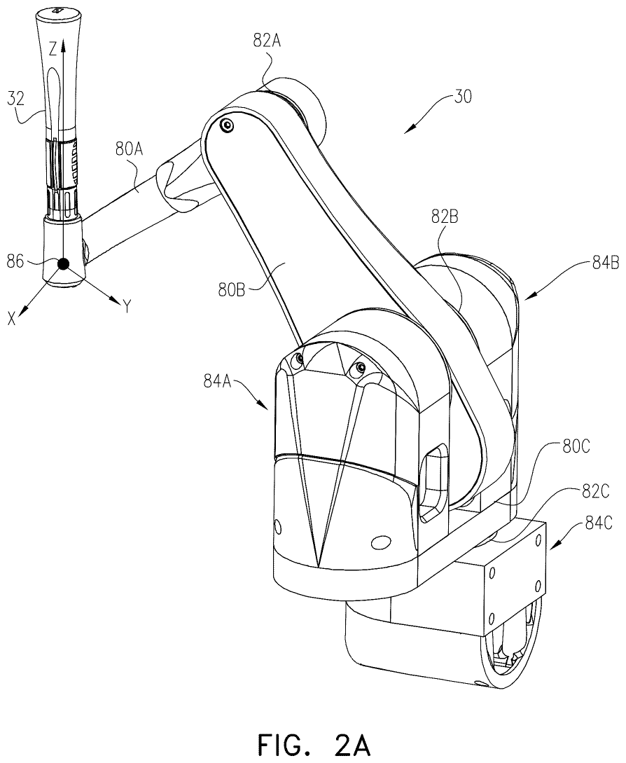

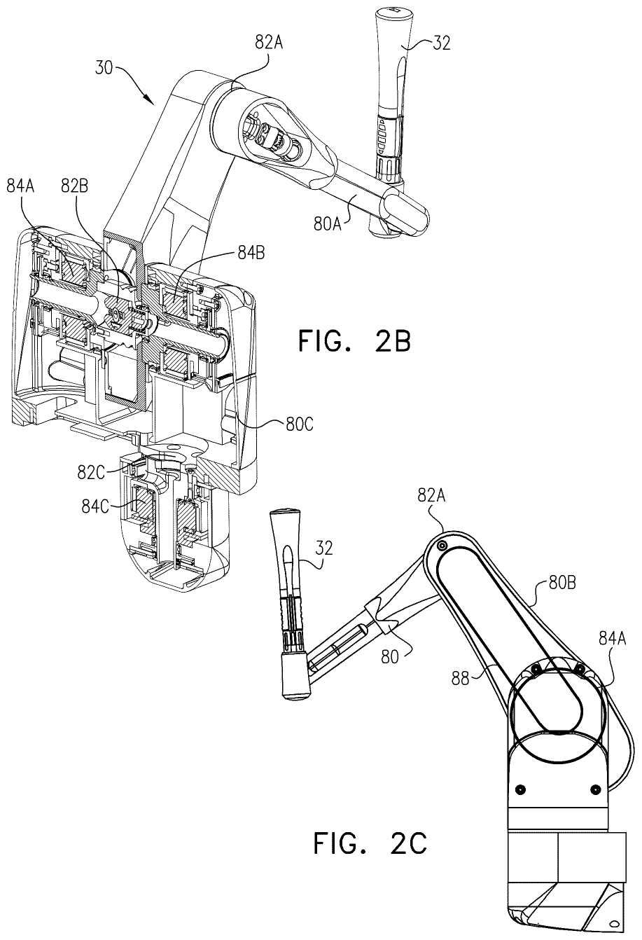

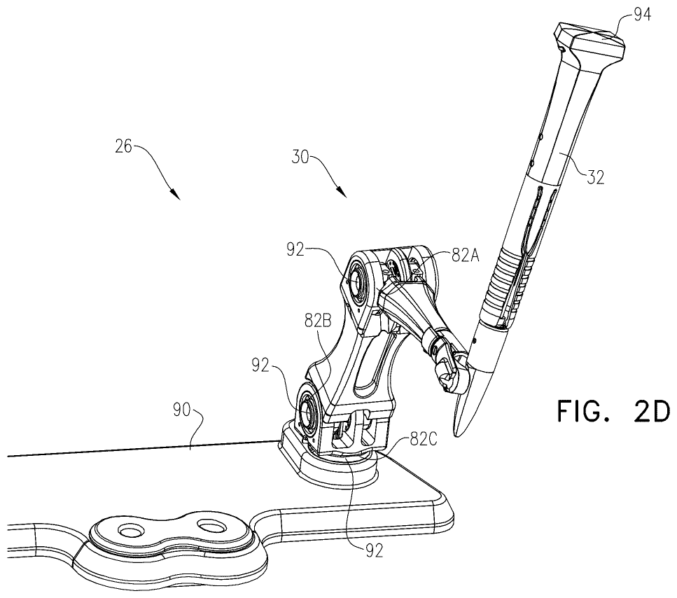

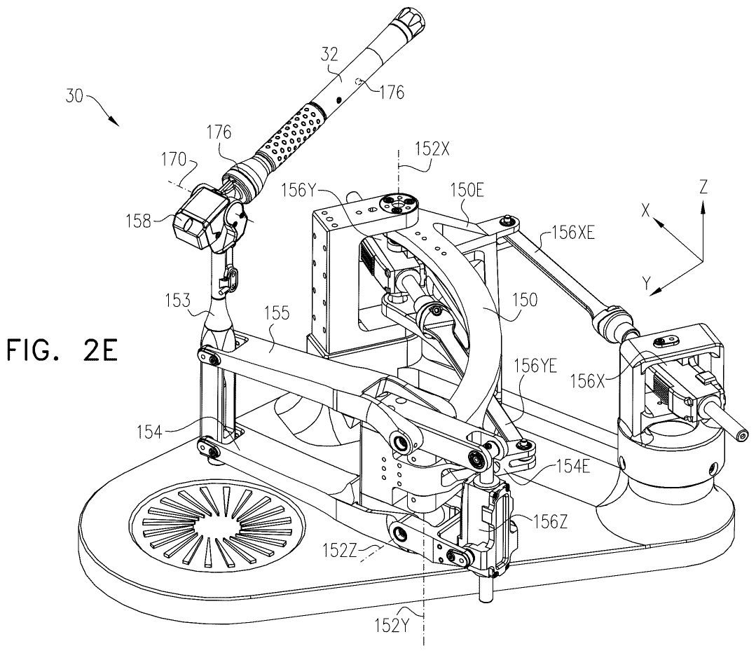

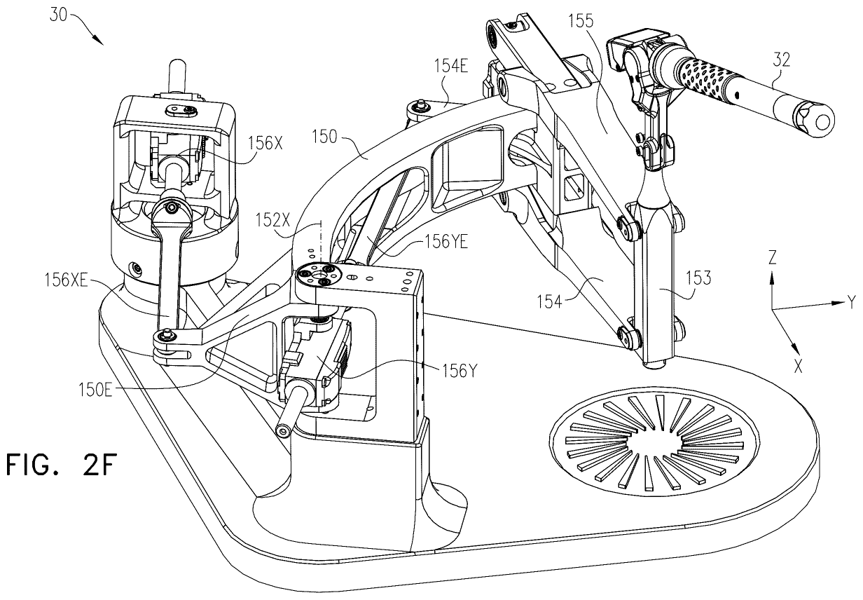

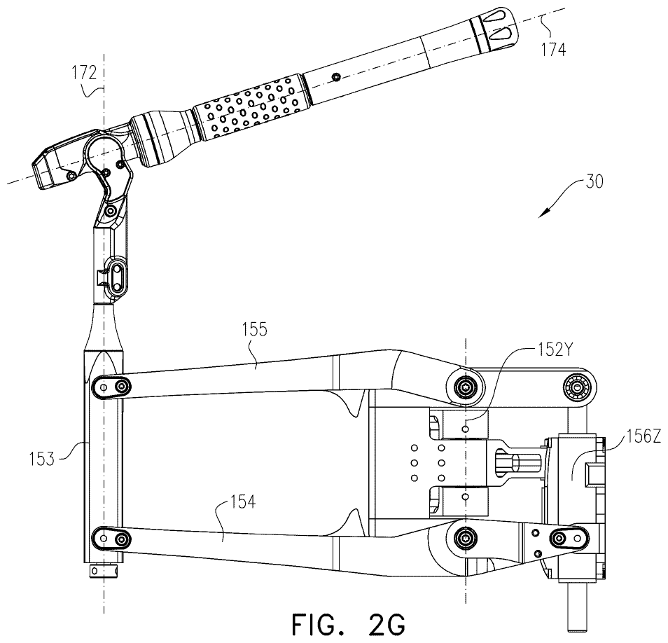

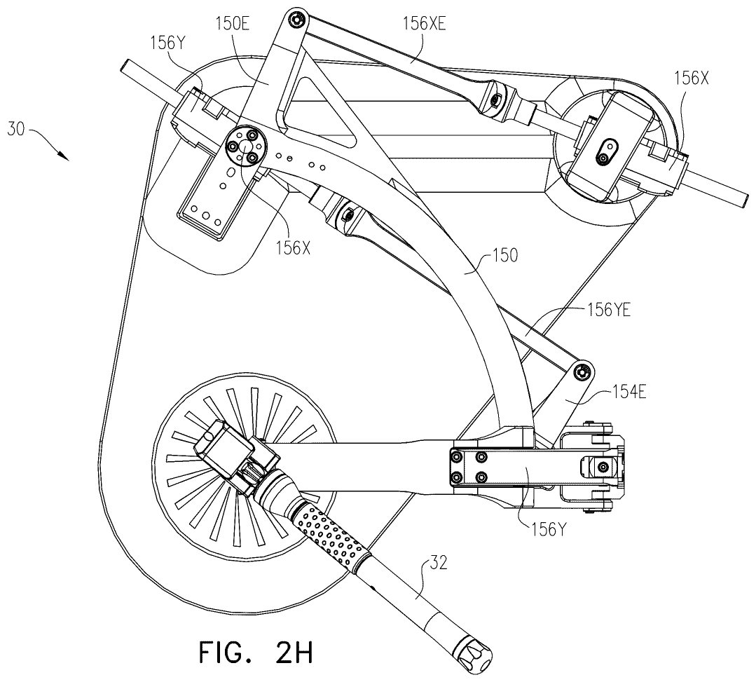

Reference is now made to FIGS. 2A, 2B, 2C, and 2D which are schematic illustrations of a control-component unit 30 that includes a control-component tool 32, in accordance with some applications of the present invention. As indicated in FIGS. 2A, 2B, and 2C, for some applications the control-component unit is configured as a control-component arm that includes two or more links, e.g., three links 80A, 80B, 80C that are connected via rotational arm joints 82A, 82B, 82C. For some applications, a respective motor 84A, 84B, 84C is configured to control movement of each of the rotational arm joints, for example, in order to provide force feedback to the operator. For some applications, at least one of the motors (84A) applies torque to one of the rotational arm joints (82A) via a belt and/or a cable 88. Typically, a belt and/or a cable is used, such that the motor can be positioned closer to a base 90 of the control-component unit (base 90 being shown in FIG. 2D), in order to reduce the weight and inertia that the operator feels, relative to if the third motor were to be placed closer to rotational arm joint 82A. For some applications, a different configuration of motors, links, and/or joints, and/or a different kinematic structure is used within the control-component unit.

Referring to FIG. 2D, typically, in addition to the above-described motors, each of the control-component arms includes a respective rotary encoder 92 coupled to each one of the rotational arm joints 82A, 82B, 82C. The rotary encoders are configured to detect movement of the respective rotational arm joints and to generate rotary-encoder data in response thereto. For some applications, the control-component arm additionally includes an inertial-measurement unit 94 that includes a three-axis accelerometer, a three-axis gyroscope, and/or a three-axis magnetometer. The inertial-measurement unit is typically disposed on the control-component tool, as shown. The rotary encoders and inertial-measurement unit are collectively referred to herein as “location sensors”. The inertial-measurement unit typically generates inertial-measurement-unit data relating to a three-dimensional orientation of the control-component arm, in response to the control-component arm being moved. For some applications, computer processor 28 receives the rotary-encoder data and the inertial-measurement-unit data. Typically, the computer processor determines the XYZ location of the tip of the control-component tool 32, based upon the rotary-encoder data, and determines the orientation of control-component tool 32 (e.g., the three Euler angles of orientation, and/or another representation of orientation) based upon the inertial-measurement-unit data, or based upon a combination of the rotary-encoder data and the inertial-measurement-unit data. Thus, based upon the rotary-encoder data and/or the inertial-measurement-unit data, the computer processor is configured to determine the XYZ location and orientation of the control-component tool. For some applications, alternative or additional types of sensors are used to determine the XYZ location and orientation of the control-component tool, such as linear encoders, rotary potentiometers, linear potentiometers, linear variable differential transformer (LVDT), cameras, magnetic sensors, etc.

Reference is now made to FIGS. 2E, 2F, 2G, and 2H, which are schematic illustrations of a control-component unit 30, in accordance with some alternative applications of the present invention. FIGS. 2E and 2F show respective oblique views of the control-component unit, FIG. 2G shows a side view, and FIG. 2H shows a top view. The functionality of control-component unit 30 as shown in FIGS. 2E-2H is generally similar to that of control-component unit 30 as shown in FIGS. 2A-2D except for the differences described hereinbelow.

Control-component unit 30 as shown in FIGS. 2E-2H typically includes a frame 150, which rotates around a first rotational axis 152X, and a link 154, which rotates around a second rotational axis 152Y and around a third rotational axis 152Z. Typically, the operator moving the control-component tool along X, Y, and Z linear directions causes the links and/or the frame to rotate around respective rotational axes. For example, as the operator moves the control-component tool along the X linear direction, this causes frame 150 to rotate about rotational axis 52X, as the operator moves the control-component tool along the Y linear direction, this causes link 154 to rotate about rotational axis 152Y, and as the operator moves the control-component tool along the Z linear direction, this causes link 154 to rotate about rotational axis 152Z.

It is noted that the above description assumes that link 154 is disposed perpendicularly to frame 150. In practice, during much of the use of the control-component unit, link 154 is disposed at an angle to frame 150. In such configurations, movement of the control-component tool within the X-Y plane (even along the X linear direction or along the Y linear direction) will typically result in both frame 150 rotating about rotational axis 152X and link 154 rotating about rotational axis 152Y. For this reason, the use of the terms X, Y, and Z as used herein in relation to movements of portions of the control-component unit should not be interpreted as strictly corresponding to movement along three linear axes that are perpendicular to each other. Rather, movement in the X and Y directions should be interpreted as relating to movement of frame 150 or link 154 within an X-Y plane (but not necessarily in directions that are perpendicular to each other) and movement in the Z direction should be interpreted as corresponding to movement of link 154 in a direction that is perpendicular to the X-Y plane. Thus, rotational axis 52X and motor 56X are associated with movement of frame 150 within the X-Y plane (regardless of whether the movement is in the X direction as indicated in the figures), rotational axis 152Y and motor 156Y are associated with movement of link 154 within the X-Y plane (regardless of whether the movement is in the Y direction as indicated in the figures), and rotational axis 152Z and motor 156Z are associated with movement of link 154 perpendicularly to the X-Y plane.

Typically, as shown, Y rotational axis 152Y is aligned with Z rotational axis 152Z along the Z direction. Further typically, both Y and Z linear motion are effected via link 154. It is noted that, for some applications, an additional supporting link 155 is disposed parallel to link 154 and rotates together with link 154. For some applications, link 154 and/or link 155 are made of two or more portions that are rigidly coupled to each other. For example, as shown in FIG. 2E, links 154 and 155 each includes a first portion disposed to the left of Z rotational axis 152Z, and a second portion disposed to the right of Z rotational axis 152Z. For some applications, a rotary encoder is disposed along each of the rotational axes 152X, 152Y, and 152Z, or a parallel rotational axis (e.g., the rotational axis of link 155). The rotary encoders detect rotation of respective links and/or frame 150 about the rotational axes, and generate signals in response thereto. The computer processor derives motion of the control-component tool along respective linear directions from the signals generated by the rotary encoders. For some applications, at least one additional rotary encoder is disposed along each of the rotational axes 152X, 152Y, and 152Z in order to provide the system with redundancy (e.g., such that in the event that one of the rotary encoders malfunctions, the other rotary encoder is used).

Typically, control-component tool 32 is moveable by the operator to undergo pitch, yaw, and roll angular rotations. The control-component tool typically undergoes pitch angular rotation by rotating about a pitch rotational axis 170, and undergoes yaw angular rotation by a shaft 153 (upon which the control-component tool is mounted) rotating about its own axis 172 (which functions as the yaw rotational axis). Typically, the control-component tool undergoes roll angular rotation by rotating about its own axis 174 (which functions as the roll rotational axis). For some applications, an inertial-measurement unit 176 is housed within the control-component tool. Typically, the inertial measurement unit includes a three-axis accelerometer, a three-axis gyroscope, and/or a three-axis magnetometer. The inertial-measurement unit typically generates inertial-measurement-unit data relating to a three-dimensional orientation of the control-component tool. Alternatively or additionally, the control-component unit includes one or more rotary encoders to detect the roll, pitch and/or yaw orientation of control-component tool 32. Typically, the rotary encoders are disposed along the axis about which the roll, pitch and yaw angular rotations occur, respectively. For some applications, the control-component unit includes inertial-measurement unit 176 in addition to one or more rotary encoders to detect the roll, pitch and/or yaw of control-component tool 32, for redundancy (e.g., such that in the event that the inertial measurement unit malfunctions, the rotary encoders are used).

Typically, computer processor 28 receives the rotary-encoder data and the inertial-measurement-unit data. Typically, the computer processor determines the XYZ location of the tip of the control-component tool 32 based upon the rotary-encoder data, and determines the three-dimensional orientation of the tip of control-component tool 32 (e.g., the three Euler angles of orientation, and/or another representation of orientation) based upon the inertial-measurement-unit data, or based upon a combination of the rotary-encoder data and the inertial-measurement-unit data. Thus, based upon the combination of the rotary-encoder data and the inertial-measurement-unit data, the computer processor is configured to determine the XYZ location and three-dimensional orientation of the tip of the control-component tool.

Typically, a direct-drive motor 156X, 156Y, 156Z (i.e., a motor that does not impart motion via gear wheels), which is typically a linear motor (e.g., a linear voice coil motor), is associated with motion along the X, Y, and Z linear directions. For some applications, the computer processor is configured to drive the control-component unit to provide force feedback to the operator that is indicative of a location of the entry of the surgical tool into the patient's eye within the incision. For some applications, the motors are configured to drive the tool to move linearly, in order to provide the aforementioned force feedback. For some applications, the computer processor is configured to apply forces that oppose the operator's attempted movements of control-component tool 32 that would result in the tool being moved beyond the edges of the incision. For example, in response to the operator moving the control-component tool through an angular yaw rotation that would cause a corresponding movement of the surgical tool that would violate the remote center of motion, the computer processor may move the control-component tool linearly (through X, Y, and/or Z linear motion) such that the remote center of motion of the surgical tool is maintained. For some such applications, the forces are applied by driving the control-component tool to move in the X, Y, and Z linear directions, via motors 156X, 156Y, 156Z.

Typically, robotic system 10 is used in procedures that require delicate and precise movements of the surgical tools, e.g., ophthalmic procedures, as described hereinabove. Therefore, control-component unit 30 is typically configured such that movement of control-component tool is performed by the operator without there being substantial counterforces to the movement (other than counterforces that are deliberately applied via motors 156X, 156Y, 156Z). For some applications, the control-component tool includes a counterweight 158, such that the weight of the control-component tool is relatively evenly balanced about pitch rotational axis 170. For some applications, the control-component tool is not entirely balanced about pitch rotational axis 170, in order to give the physician a feeling of the tool's weight (like a real surgical tool), and/or to reduce the overall mass of the control-component tool. For some applications, link 154 extends across both sides of Z rotational axis 152Z, with the control-component tool and additional components being disposed on link 154 (and/or parallel link 155) on a first side of rotational axis 152Z. For some applications, motor 156Z, which is disposed along the Z linear direction, is disposed on link 154 on the other side of rotational axis 152Z, such as to balance the weight of the control-component tool and additional components that are disposed on the first side. For some such applications, the control-component unit does not include an additional counterweight for this purpose. Alternatively, the control-component unit includes a counterweight for this purpose, in addition to motor 156Z.

For some applications, frame 150 (which functions as the link through which X direction linear motion is effected) comprises two curved arms and motor 156Y (and, optionally, an extension 156YE thereof) passes between the two curved arms along a straight line. For some applications, an end of frame 150 which is adjacent to Z rotational axis 152Z is aligned with Z rotational axis 152Z (as shown in FIG. 2E), such that frame 150 does not exert any torque about Z rotational axis 152Z. Thus, frame 150 does not need to be counterbalanced about Z rotational axis 152Z. For some applications, even as frame 150 moves (due to motion in the X direction), the frame remains aligned with Z rotational axis 152Z, such that no compensatory motion is necessary in order to balance the motion of the frame.

It is noted that in accordance with the above description, the control-component unit typically is balanced within all six degrees of freedom (the three axial translations and three angular rotations). For some applications, the control-component unit utilizes counterweights to provide balance in two degrees of freedom: Z direction axial motion and pitch angular motion. In the embodiment shown in FIGS. 2E-2H, motor 156Z functions as the counterweight in the Z direction axial motion degree of freedom. The remaining four degrees of freedom (i.e., X and Y axial motion, and roll and yaw angular motions) typically do not require counterweights for balancing, since the control-component unit is designed such that the control-component tool and/or other elements of the control-component unit are self-balancing within these degrees of freedom. Since the control-component unit is designed to be balanced within all six degrees of freedom (e.g., by self-balancing within four degrees of freedom and balance being provided by the counterweights within the two remaining degrees of freedom), the control-component tool tends to maintain its position and orientation, in the absence of any forces acting upon the control-component tool. Thus, typically if the operator temporarily lets go of the control-component tool (without exerting force on the control-component tool as she/he lets go of the tool), the control-component tool maintains its position and orientation until the operator resumes control of the control-component tool. Further typically, the control-component tool is able to provide force feedback to the operator at relatively low levels of force, since the control-component tool provides relatively low inertial forces. I.e., the motors that are configured to provide force feedback to the operator by driving the control-component tool to move are configured to do so substantially without being required to overcome inertial forces.

For some applications, as shown in FIGS. 2E-2H, motor 156Y is disposed within the X-Y plane such that its center of mass is substantially aligned with X rotational axis 152X both when motor 156Y is extended and when motor 156Y is retracted. Typically, this prevents movement of motor 156Y from exerting any torque in the Z direction on link 154 as motor 156Y extends and retracts. It is noted that as the motor extends and retracts, its center of mass moves slightly. Typically, the motor is positioned such that in at least one position within its fully extended and fully retracted states, the motor's center of mass is aligned with X rotational axis 152X. Further typically, the motor's center of mass is aligned with X rotational axis 152X, when the motor is at its central position with respect to its fully extended and fully retracted states. For some applications, both when the motor is fully extended and fully retracted, its center of mass is within 10 mm, e.g., within 5 mm of X rotational axis 152X. It is further noted that motor 156Y is typically coupled to frame 150, such that motor 156Y is configured to rotate together with frame 150. By being configured in this manner, the motor does not apply any torque to frame 150 even as frame 150 rotates.

For some applications, frame 150 includes an angled extension 150E to which motor 156X (and, optionally, an extension 156XE thereof) is coupled. Motor 156X rotates frame 150 about axis 152X by the motor (or the extension thereof) pushing or pulling angled extension 150E. Typically, by the control-component unit incorporating angled extension 150E, the dimensions of the control-component unit (and the overall footprint of the control component) are reduced relative to if motor 156X (or extension 156XE thereof) were to be coupled to a non-angled continuation of frame 150 on an opposite side of axis 152X from the main portion of frame 150. For some applications (not shown), motor 156X (or the extension thereof) rotates frame 150 about axis 52X by pushing or pulling a non-angled extension that is disposed within the footprint of the frame.

Similarly, for some applications, link 154 includes an angled extension 154E to which motor 156Y (and, optionally, an extension 156YE thereof) is coupled. Motor 156Y (or the extension thereof) rotates link 154 about axis 152Y by pushing or pulling angled extension 154E. Typically, by the control-component unit incorporating angled extension 154E, the dimensions of the control-component unit (and the overall footprint of the control component) are reduced relative to if motor 156Y (or extension 156YE thereof) were to be coupled to a non-angled continuation of link 154 on an opposite side of axis 152Y from the main portion of link 154. For some applications (not shown), motor 156Y (or the extension thereof) rotates frame 150 about axis 152Y by pushing or pulling link 154 at location that is offset from the Y rotational axis 152Y.

For some applications, longitudinal axis 172 of shaft 153 (which functions as the yaw rotational axis) is aligned with the ends of links 154 and 155.

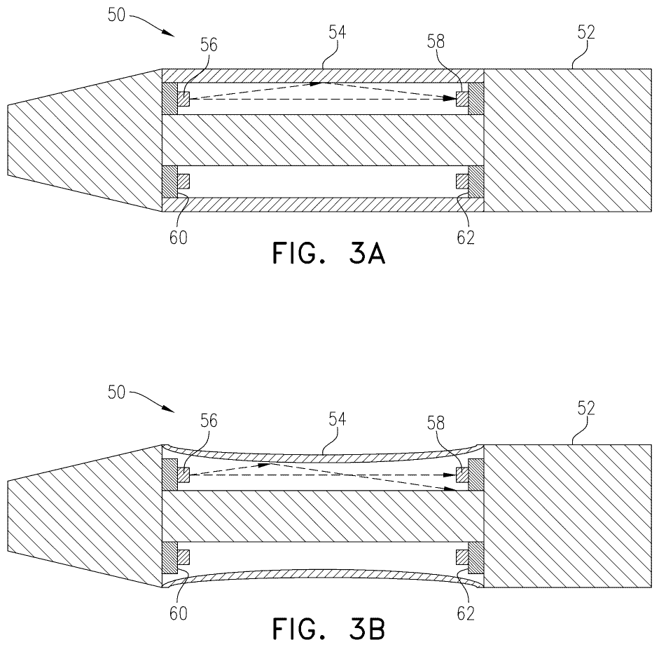

As described hereinabove, for some applications, the operator provides commands to the robotic units via control component 26. Typically, such commands include commands that control actions that are performed by the tools. For example, the commands may control a blade, a phacoemulsification tool (e.g., the operation mode and/or suction power of the phacoemulsification tool), forceps (e.g., opening and closing of forceps), an intraocular-lens-manipulator tool (e.g., such that the tool manipulates an intraocular lens inside the eye for precise positioning of the intraocular lens within the eye), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, orientation, and/or XYZ positioning of the imaging system). For some applications, control-component tool 32 (and/or a different portion of the control-component unit) includes one or more input-receiving components 50 that are configured to receive such inputs from the operator. Input-receiving components 50 in accordance with some applications of the present invention are shown in FIGS. 3A-5C and 8A-10B.

For some applications, input-receiving component 50 is configured to be rotationally agnostic, such that the input that is detected is the same regardless of the roll orientation of the control-component tool relative to the operator's hand. In some cases, this is desirable, since the control-component tool typically undergoes roll angular rotation relative to the operator's hand during use. For some applications, the one or more input-receiving components are disposed at discrete circumferential positions with respect to the control-component tool, such that the input-receiving components can only be pressed at certain circumferential positions. In some cases this is desirable, in order for the input-receiving components to more closely resemble conventional surgical tools such as forceps.

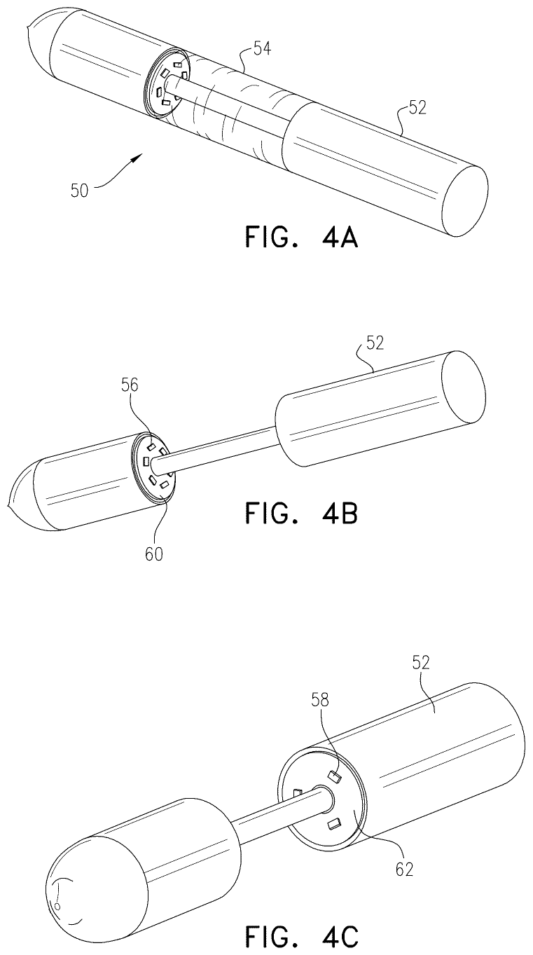

Reference is now made to FIGS. 3A and 3B, which are schematic illustrations of a cross-sectional view of a handle 52 of a control-component tool 32 that includes input-receiving component 50, in accordance with some applications of the present invention. Reference is also made to FIGS. 4A, 4B and 4C, which are schematic illustrations of portions of the handle of the control-component tool of FIGS. 3A and 3B, in accordance with some applications of the present invention. For some applications, input-receiving component 50 includes a deformable sleeve 54, one or more light sources 56, and one or more light detectors 58. For some applications, the deformable sleeve comprises an elastomeric material, such as silicone. For some applications, handle 52 defines a first radial wall 60 and the one or more light sources include a plurality of LEDS which are positioned circumferentially upon the first radial wall such that the LEDs direct light parallel to the axis of the handle. For some applications, handle 52 defines a second radial wall 62 that faces the first radial wall, and the one or more light detectors include one more ambient light detectors that are disposed circumferentially upon the second radial wall such that the light detectors face the LEDs. Typically, the deformable sleeve extends axially between the first and second radial walls.

For some applications, the control-component unit is configured such that the operator provides an input to the robotic system by pressing deformable sleeve 54. Typically, the pressure on the deformable sleeve causes the sleeve to deform (as shown in the transition from FIG. 3A to FIG. 3B), and at least partially attenuate light that is directed from light sources 56 toward light detectors 58. Computer processor 28 typically receive a signal that is indicative of the light attenuation from the light detectors and controls the robotic system and/or the imaging system in response to the detected light attenuation. For some applications, based upon the level and/or type of light attenuation, the computer processor detects the extent to which the sleeve has been deformed (which is indicative of the amount of pressure that has been applied by the operator) and controls the robotic system and/or the imaging system in response thereto. For example, the amount of force with which forceps are closed by the robotic system may be controlled in response to the amount of pressure that has been applied by the operator. For some applications, input-receiving component 50 is configured to receive an input via pressure that is applied to the deformable sleeve by a single finger or thumb of the operator. Alternatively or additionally, input-receiving component 50 is configured to receive an input via pressure that is applied to the deformable sleeve by two fingers (or a finger and a thumb), for example in a squeezing action.

As noted above, for some applications, input-receiving component 50 is configured to be rotationally agnostic, such that the input that is detected is the same regardless of the roll orientation of the control-component tool relative to the operator's hand. Typically, for such applications, the light sources and light detectors are distributed uniformly around the circumference of handle 52. For some applications, the light sources and light detectors are distributed non-uniformly around the circumference of handle 52 and the control component is configured for the operator to provide an input by applying pressure to the deformable sleeve at discrete locations around the circumference of handle 52.

It is noted that input-receiving component 50 does not rely upon rigid mechanical motion of any components in order for the computer processor to detect an input by the operator. Thus, the input-receiving component is not susceptible to mechanical breakdown that is typical of components that do rely upon such rigid mechanical motion.

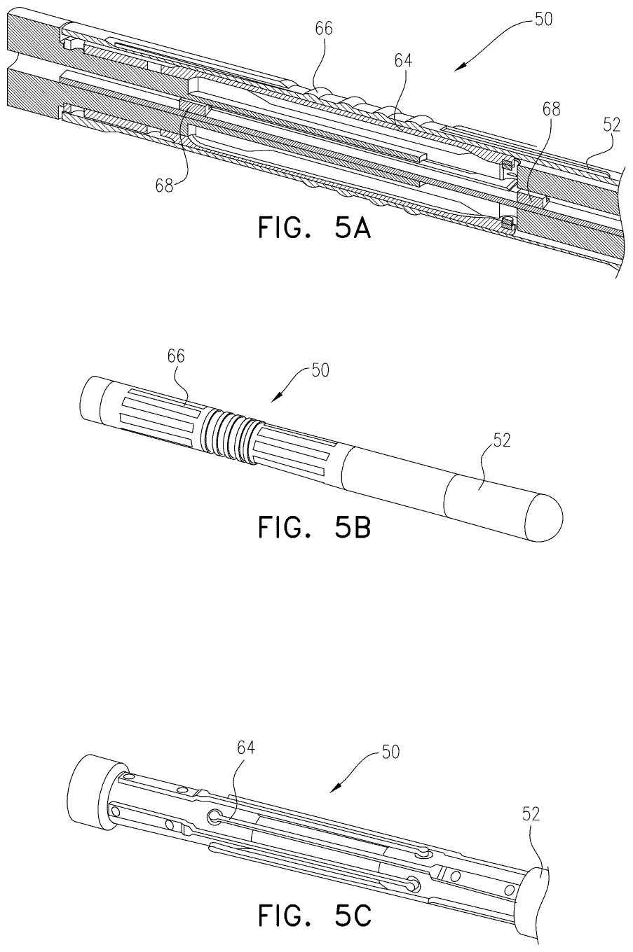

Reference is now made to FIGS. 5A, 5B, and 5C which are schematic illustrations of a cross-sectional view of a handle 52 of a control-component tool 32 that includes input-receiving component 50, in accordance with some applications of the present invention. For some applications, the input-receiving component includes one or more magnetic structures 64 (e.g., magnetic leaf springs arranged in parallel with the axis of the handle). Typically, the magnetic structures are covered with a flexible sleeve 66, such as an elastomeric sleeve (e.g., a silicone sleeve). For some applications, one or more magnetic materials are embedded within sleeve 66. For some applications, the sleeve itself is magnetic. For example, the sleeve may include magnetic silicone. Typically, one or more magnetometers 68 (e.g., Hall sensors) are disposed within handle 52 and are configured to measure magnetic flux that is generated by the magnetic structures in response to pressure being applied to the magnetic structures by the operator.

Computer processor 28 typically controls the robotic system and/or the imaging system in response to the detected magnetic flux that is generated by the magnetic structures. For some applications, the computer processor detects the amount of pressure that has been applied by the operator and controls the robotic system and/or the imaging system in response thereto. For example, the amount of force with which forceps are closed by the robotic system may be controlled in response to the amount of pressure that has been applied by the operator. For some applications, input-receiving component 50 is configured to receive an input via pressure that is applied to the magnetic structures by a single finger or thumb of the operator. Alternatively or additionally, input-receiving component 50 is configured to receive an input via pressure that is applied to the magnetic structures by two fingers (or a finger and a thumb), for example in a squeezing action.

For some applications, input-receiving component 50 is configured to be rotationally agnostic, such that the input that is detected is the same regardless of the roll orientation of the control-component tool relative to the operator's hand. Typically, for such applications, the magnetic structures are distributed uniformly around the circumference of handle 52. For some applications, the magnetic structures are distributed non-uniformly around the circumference of handle 52 and the control component is configured for the operator to provide an input by applying pressure to the deformable sleeve at discrete locations around the circumference of handle 52.

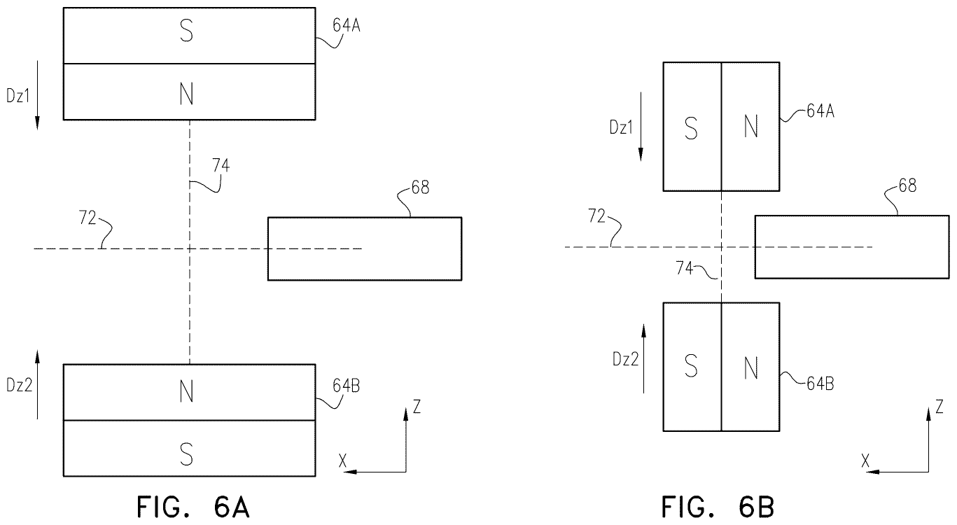

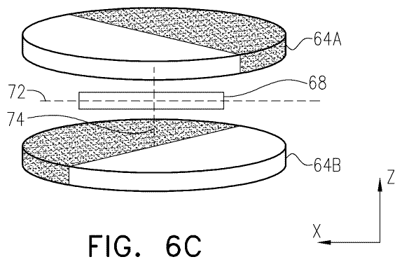

Reference is now made to FIGS. 6A, 6B, and 6C which are schematic illustrations of respective arrangements of magnetic structures 64 and magnetometer 68, in accordance with some applications of the present invention. Typically, magnetometer 68 is a three-dimensional magnetometer. Referring to FIG. 6A, for some applications, at one or more circumferential locations around the handle the magnetic structures include two magnets 64A and 64B disposed on radially opposite sides of the handle from each other. The magnets are arranged with their magnetic axes perpendicular to the longitudinal axis of the handle, and with the magnets facing in opposite directions from each other, such that the same pole of each of the magnets is disposed closer to the radial center (i.e., the longitudinal axis) of the handle. For example, as shown in FIG. 6A, the North pole of each of the magnets is disposed closer to the radial center of the handle than the South pole. For some applications, the magnetometer is disposed within the handle along the radial centerline 72 of the two magnets (i.e., the centerline as measured across the radial direction of the handle, which is also the longitudinal axis of the handle) but offset from the axial centerline 74 of the magnets (i.e., the centerline of the magnets as measured along the axial direction of the handle).

Referring to FIG. 6B, for some applications, at one or more circumferential locations around the handle the magnetic structures include two magnets 64A and 64B disposed on radially opposite sides of the handle from each other, with their magnetic axes parallel to the longitudinal axis of the handle and with their poles oriented in the same direction as each other. As with the configuration shown in FIG. 6A, the magnetometer is typically disposed within the handle along the radial centerline 72 of the two magnets but offset from the axial centerlines 74 of the magnets.

Referring to FIG. 6C, for some applications, at one or more circumferential locations around the handle the magnetic structures include two magnets 64A and 64B disposed on radially opposite sides of the handle from each other, with their magnetic axes disposed within the x-y plane, and with their poles in different, but not opposite, orientations from each other (i.e., not at 0 degrees and not at 180 degrees from each other). For some such applications, the magnetometer is disposed within the handle along radial centerline 72 of the two magnets and along axial centerline 74 of the magnets.

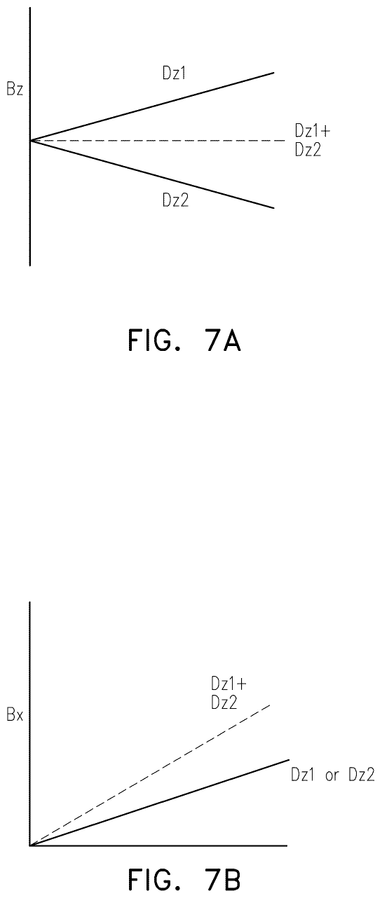

Reference is now made to FIGS. 7A and 7B, which are graphs indicating the magnetic flux that is generated along respective directions using the arrangement shown in FIG. 6A, in accordance with some applications of the present invention. FIG. 7A indicates that in response to either magnet 64A being pushed radially inwardly (indicated by Dz1 in FIG. 6A) or magnet 64B being pushed radially inwardly (indicated by Dz2 in FIG. 6A), then equal but opposite magnetic flux along the radial direction (indicated by Bz) is generated. Thus, if just one of the magnets is pushed radially inward (e.g., by a single finger or thumb of the operator), the computer processor is able to determine which of the magnets was pushed and by how much, based on the magnetic flux along the radial direction. However, if both magnets 64A and 64B are pushed radially inwardly, the magnetic flux along the radial direction (indicated by Bz) generated by each of the magnets cancel each other. (For example, if both magnets are pushed radially inwardly by the same distance as each other, then the net magnetic flux that is generated is zero, as indicated by the dashed line in FIG. 7A.) Thus, when both magnets 64A and 64B are pushed radially inwardly, the computer processor is unable to determine the pressure with which each one of the magnets has been pushed.

FIG. 7B indicates that in response to either magnet 64A being pushed radially inwardly (indicated by Dz1 in FIG. 6A) or magnet 64B being pushed radially inwardly (indicated by Dz2 in FIG. 6A), magnetic flux along the axial direction (indicated by Bx) is generated. This is due to the axial offset of the magnetometer from the axial center line of the magnets. Furthermore, typically, in response to either magnet 64A being pushed radially inwardly (indicated by Dz1 in FIG. 6A) or magnet 64B being pushed radially inwardly (indicated by Dz2 in FIG. 6A), the magnetic flux along the axial direction (indicated by Bx) that is generated is in the same direction. Therefore, if both magnets 64A and 64B are pushed radially inwardly, the magnetic flux along the axial direction (indicated by Bx) generated by each of the magnets is added to each other. Typically, based upon the magnetic flux measured along both the radial and axial directions, the computer processor is able to determine the total pressure with which the magnets have been pressed.

The scope of the present application includes other variations of configurations of magnets and magnetometers (including other configurations of magnets, magnetometer location, magnetometer size, and/or magnetometer orientation). Typically, the configurations are such that the computer processor is able to derive the total force with which the input-receiving component 50 has been pressed. For some applications, the configurations are such that the computer processor is able to derive the circumferential location(s) at which input-receiving component 50 has been pressed. For some applications, the magnets and magnetometer are arranged as shown in FIG. 6B and/or as shown in FIG. 6C, such that the computer processor is able to distinguish between when the first magnet is pressed, the second magnet is pressed, or both magnets are pressed, since each of these inputs will give rise to a different change in the magnetic flux that is detected at the magnetometer.

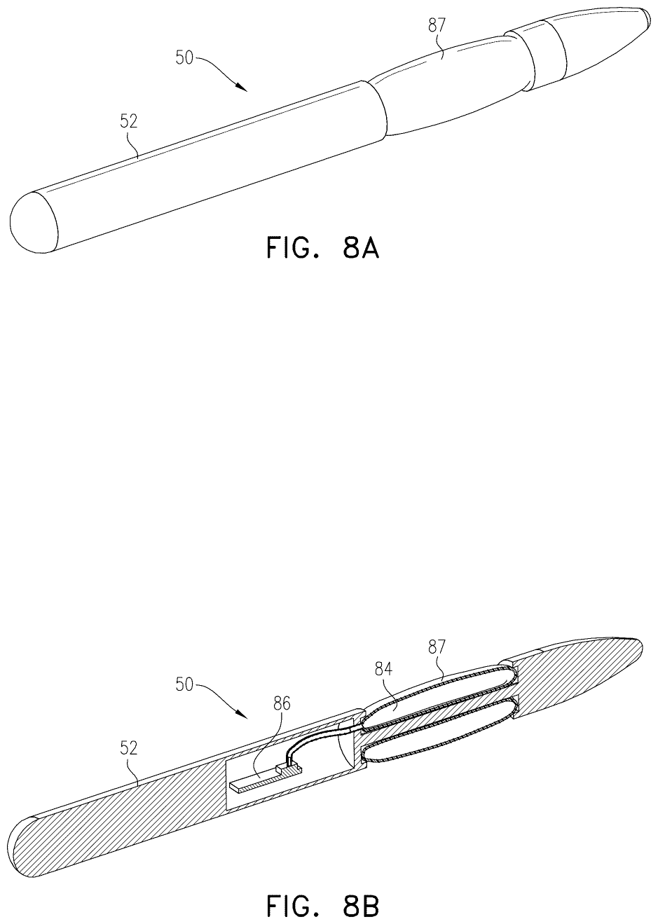

Reference is now made FIGS. 8A and 8B, which are schematic illustrations of an oblique view and a cross-sectional view of a handle 52 of a control-component tool 32 that includes input-receiving component 50, in accordance with some applications of the present invention. For some applications, input-receiving component 50 includes a fluid-filled chamber 84 that is coupled to a pressure sensor 86. For example, the fluid-filled chamber may include an elastomeric sleeve 87 that houses air, oil, or a different fluid. Typically, in response to the operator pressing the fluid-filled chamber, the pressure within the fluid-filled chamber changes, and the pressure sensor senses the change in pressure.

Computer processor 28 typically controls the robotic system and/or the imaging system in response to the detected change in pressure. For some applications, the computer processor detects the amount of pressure that has been applied by the operator and controls the robotic system and/or the imaging system in response thereto. For example, the amount of force with which forceps are closed by the robotic system may be controlled in response to the amount of pressure that has been applied by the operator. For some applications, input-receiving component 50 is configured to receive an input via pressure that is applied to the fluid-filled chamber by a single finger or thumb of the operator. Alternatively or additionally, input-receiving component 50 is configured to receive an input via pressure that is applied to the fluid-filled chamber by two fingers (or a finger and a thumb), for example in a squeezing action.

For some applications, input-receiving component 50 is configured to be rotationally agnostic, such that the input that is detected is the same regardless of the roll orientation of the control-component tool relative to the operator's hand and/or regardless of whether pressure is applied using two fingers (or a finger and a thumb) or a single finger or thumb. Typically, for such applications, there is a single fluid-filled chamber encompassing the full circumference of handle 52. For some applications, a plurality of fluid-filled chambers are distributed around the circumference of the handle and the computer processor, based on signals from the pressure sensor (or a plurality of pressure sensors), is configured to detect which of the fluid-filled chambers were pressed, and thereby determine which and how many circumferential locations were pressed.

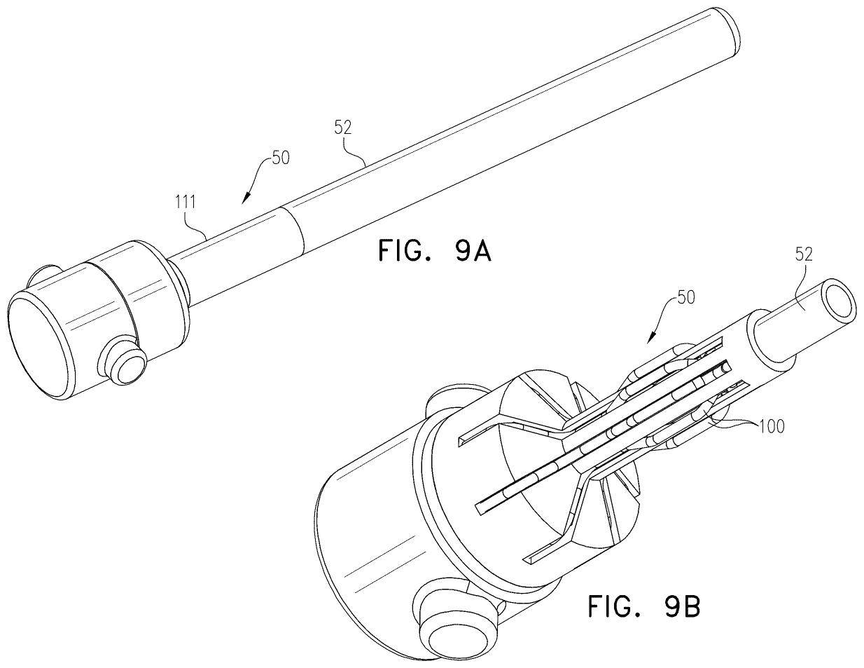

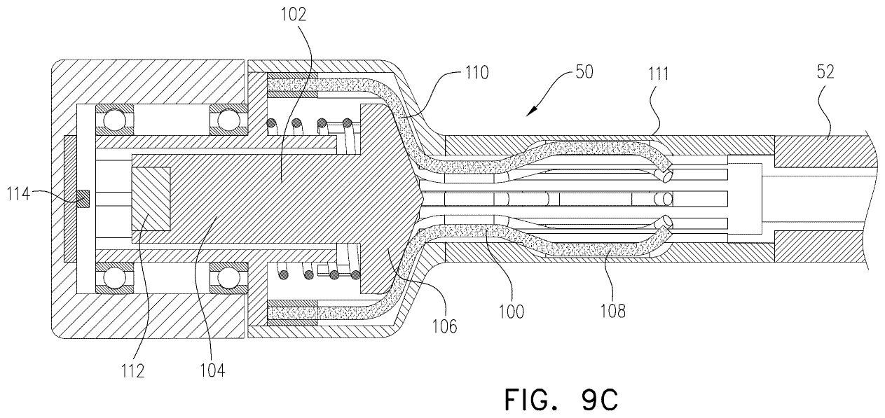

Reference is now made to FIGS. 9A, 9B, and 9C, which are schematic illustrations of portions a handle 52 of a control-component tool 32 that includes input-receiving component 50, in accordance with some applications of the present invention. For some applications, input-receiving component 50 includes one or more spring wires 100, and a piston barrel 102 with a neck 104 and a conical head 106 that is typically wider than the neck. Each of the spring wires includes a first radially-protruding portion 108 (which typically protrudes from a housing of the handle) and a second portion 110 that contacts the conical head of the piston barrel. For some applications, the radially-protruding portions of the spring wires are covered with a sleeve 111, for example an elastomeric sleeve, such as a silicone sleeve. (Handle 52 is shown in the absence of sleeve 111 in FIG. 9B, for illustrative purposes.) Typically, in response to the radially-protruding portion of even one of the spring wires being pressed, the second portion of the wire pushes the piston barrel axially. Typically, a magnet 112 is disposed within neck 104 of the piston barrel, and a magnetometer 114 is configured to detect the axial motion of the piston barrel.

Computer processor 28 typically controls the robotic system and/or the imaging system in response to the detected axial motion of the piston barrel. For some applications, the computer processor detects the amount of pressure that has been applied by the operator and controls the robotic system and/or the imaging system in response thereto. For example, the amount of force with which forceps are closed by the robotic system may be controlled in response to the amount of pressure that has been applied by the operator. For some applications, input-receiving component 50 is configured to receive an input via pressure that is applied to the spring wires by a single finger or thumb of the operator. Alternatively or additionally, input-receiving component 50 is configured to receive an input via pressure that is applied to the spring wires by two fingers (or a finger and a thumb), for example in a squeezing action.

For some applications, input-receiving component 50 is configured to be rotationally agnostic, such that the input that is detected is the same regardless of the roll orientation of the control-component tool relative to the operator's hand and/or regardless of whether pressure is applied using two fingers (or a finger and a thumb) or a single finger or thumb. Typically, for such applications, the spring wires are distributed uniformly around the full circumference of handle 52. For some applications, spring wires are distributed non-uniformly around the circumference of handle 52, and the computer processor is configured to detect which circumferential locations were pressed by the operator.

For some applications, piston barrel 102 is coupled to handle 52, such that roll angular rotation of the handle causes the piston barrel to roll. For some applications, magnetometer 114 (and/or a different magnetometer) detects roll angular rotation that magnet 112 undergoes, and the computer processor thereby determines that the handle has undergone roll angular rotation. For some applications, one or more additional sensors are configured to detect axial and/or rotational motion of the piston barrel. For example, one or more vibration sensors, accelerometers, and/or inertial measurement units may be disposed within the handle and configured to detect axial and/or rotational motion of the piston barrel.

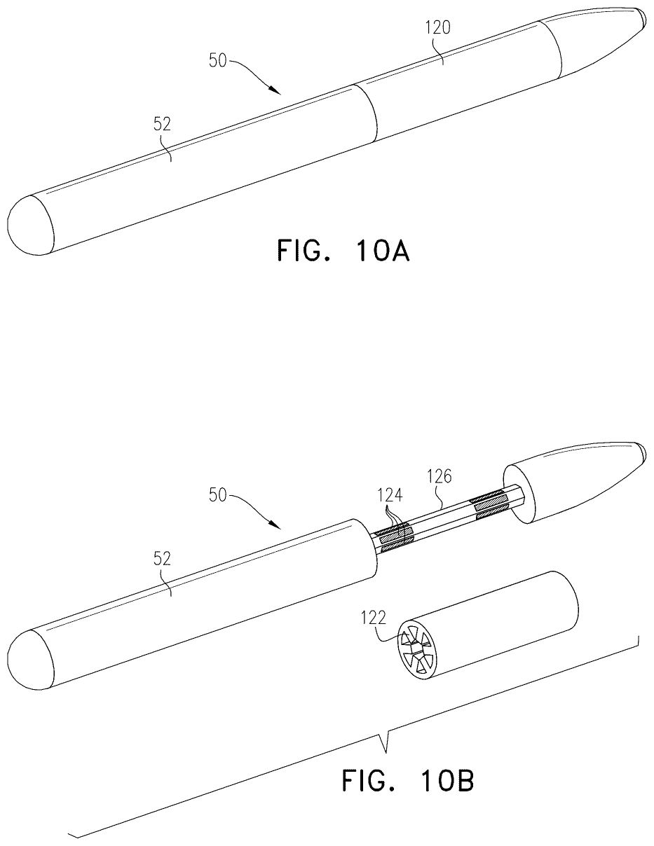

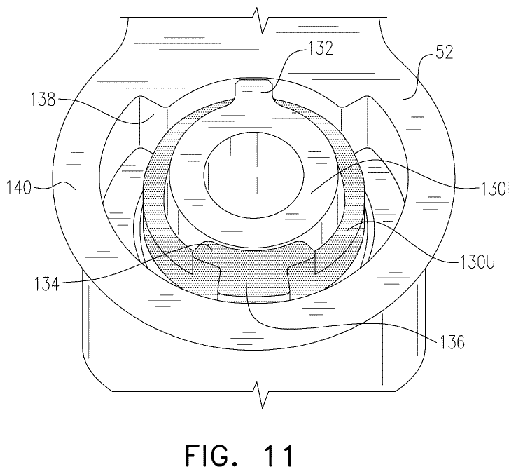

Reference is now made to FIGS. 10A and 10B, which are schematic illustrations of portions a handle 52 of a control-component tool 32 that includes input-receiving component 50, in accordance with some applications of the present invention. For some applications, input-receiving component 50 includes a sleeve 120 from which a plurality of ribs 122 extend radially inwardly. A plurality of pressure sensors 124 are disposed along an inner shaft 126 that is concentric with sleeve 120 and inside the sleeve. In response to the operator pressing the sleeve, at least one of the ribs is pressed against a pressure sensor, and the pressure generated by the rib is detected by the pressure sensor.

Computer processor 28 typically controls the robotic system and/or the imaging system in response to the pressure detected by pressure sensors 124. For some applications, the computer processor detects the amount of pressure that has been applied by the operator and controls the robotic system and/or the imaging system in response thereto. For example, the amount of force with which forceps are closed by the robotic system may be controlled in response to the amount of pressure that has been applied by the operator. For some applications, input-receiving component 50 is configured to receive an input via pressure that is applied to sleeve 120 by a single finger or thumb of the operator. Alternatively or additionally, input-receiving component 50 is configured to receive an input via pressure that is applied to sleeve 120 by two fingers (or a finger and a thumb), for example in a squeezing action.

For some applications, the input-receiving component 50 is configured to be rotationally agnostic, such that the input that is detected is the same regardless of the roll orientation of the control-component tool relative to the operator's hand and/or regardless of whether pressure is applied using two fingers (or a finger and a thumb) or a single finger or thumb. Typically, for such applications, ribs 122 and pressure sensors 124 are distributed uniformly around the full circumference of handle 52. For some applications, ribs 122 and pressure sensors 124 are distributed non-uniformly around the circumference of handle 52, and the computer processor is configured to detect which circumferential locations were pressed by the operator.

Reference is now made to FIG. 11, which is a schematic illustration of a mechanism for controlling the roll angular rotation of a control-component tool, in accordance with some applications of the present invention. For some applications, it is desirable to permit roll angular rotation of the control-component tool over more than 360 degrees. For some such applications, inner and outer roll-limiting components (130I and 130U) are disposed within handle 52 of the control-component tool, with the inner roll-limiting component disposed within the outer roll-limiting component. For some applications, the inner roll-limiting component has a circular cross-section with a radial protrusion 132. Roll of the inner roll-limiting component is limited by radial protrusion 132 being blocked by an inner radial protrusion 134 from the outer roll-limiting component. For some applications, the outer roll-limiting component has a circular cross-section with inner radial protrusion 134 and an outer radial protrusion 136. Roll of the outer roll-limiting component is limited by radial protrusion 136 being blocked by an inner radial protrusion 138 from an inner surface of the housing 140 of handle 52. For some applications, the total amount of roll angular rotation that is permitted by rolling both the inner and outer roll-limiting components is more than 400 degrees (e.g., more than 450 degrees) and/or less than 600 degrees (e.g., less than 550 degrees), e.g., between 400 and 600 degrees, or between 450 and 550 degrees.

For some applications, as an alternative or in addition to the mechanism shown in FIG. 11, the control-component tool includes one or more slip rings, in order to allow the control-component tool to roll through more than 360 degrees, while providing power to, and receiving data from, the control-component tool, via wiring. For some applications, by including one or more slip rings, the control-component tool can be rolled around it longitudinal axis in a non-limited manner.

Although some applications of the present invention are described with reference to ophthalmic surgery, the scope of the present application includes applying the apparatus and methods described herein to other medical procedures, mutatis mutandis. In particular, the apparatus and methods described herein to other medical procedures may be applied to other microsurgical procedures, such as general surgery, orthopedic surgery, gynecological surgery, otolaryngology, neurosurgery, oral and maxillofacial surgery, plastic surgery, podiatric surgery, vascular surgery, and/or pediatric surgery that is performed using microsurgical techniques. For some such applications, the imaging system includes one or more microscopic imaging units.

It is noted that the scope of the present application includes applying the apparatus and methods described herein to any ophthalmic procedure. Such procedures may include cataract surgery, collagen crosslinking, endothelial keratoplasty (e.g., DSEK, DMEK, and/or PDEK), DSO (descemet stripping without transplantation), laser assisted keratoplasty, keratoplasty, LASIK/PRK, SMILE, pterygium, ocular surface cancer treatment, secondary IOL placement (sutured, transconjunctival, etc.), iris repair, IOL reposition, IOL exchange, superficial keratectomy, Minimally Invasive Glaucoma Surgery (MIGS), limbal stem cell transplantation, astigmatic keratotomy, Limbal Relaxing Incisions (LRI), amniotic membrane transplantation (AMT), glaucoma surgery (e.g., trabs, tubes, minimally invasive glaucoma surgery), automated lamellar keratoplasty (ALK), anterior vitrectomy, and/or pars plana anterior vitrectomy.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described with reference to the Figures, computer processor 28 typically acts as a special purpose robotic-system computer processor. Typically, the operations described herein that are performed by computer processor 28 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus for performing a procedure on a patient using a surgical tool, the apparatus comprising:
a robotic unit configured to control the surgical tool; and
a control-component tool that is configured to be held an operator, the control-component tool comprising:
a handle; and
an input-receiving component disposed on the handle that comprises:
a first radial wall with one or more light sources disposed on the first radial wall;
a second radial wall that faces the first radial wall, with one or more light detectors disposed on the second radial wall;
and a deformable sleeve extending axially between the first and second radial walls and configured to attenuate light that is directed from the one or more light sources toward the one or more light detectors, in response to being pressed; and
a computer processor configured to:
receive a signal from the one or more light detectors that is indicative of the light attenuation; and
control the surgical tool in response thereto.

2. The apparatus according to claim 1, wherein the input-receiving component does not rely upon rigid mechanical motion of any components in order for the computer processor to detect an input to the input-receiving component.

3. The apparatus according to claim 1, wherein the input-receiving component is configured to receive an input via pressure that is applied to the deformable sleeve by a single finger or thumb of an operator.

4. The apparatus according to claim 1, wherein the input-receiving component is configured to receive an input via pressure that is applied to the deformable sleeve by two fingers.

5. The apparatus according to claim 1, wherein the light sources and light detectors are distributed non-uniformly around a circumference of the handle and the attenuation of the light in response to the deformable sleeve being pressed only occurs at discrete locations around the circumference of handle.

6. The apparatus according to claim 1, wherein the apparatus is configured for performing an ophthalmic procedure on an eye of a patient using one or more surgical tools that have tips and wherein the robotic unit is configured to move the one or more surgical tools within the patient's eye.

7. The apparatus according to claim 6, wherein the computer processor is configured to:
determine movement of the location and orientation of the tip of the control-component tool based upon data received from the one or more location sensors; and
move the tip of the surgical tool within the patient's eye in a manner that corresponds with the movement of the location and orientation of the tip of the control-component tool.

8. The apparatus according to claim 1, wherein the input-receiving component is configured to be rotationally agnostic, such that the attenuation of the light in response to the deformable sleeve being pressed is the same regardless of a roll orientation of the control-component tool relative to an operator's hand.

9. The apparatus according to claim 8, wherein the light sources and light detectors are distributed uniformly around a circumference of handle.

10. An apparatus for performing a procedure on a patient using a surgical tool, the apparatus comprising:
a robotic unit configured to control the surgical tool; and
a control-component tool that is configured to be held by an operator, the control-component tool comprising:
a handle; and
an input-receiving component disposed on the handle that comprises:
one or more magnetic leaf springs; and
a magnetometer; and
a computer processor configured to:
receive a signal from the magnetometer that is indicative of the one or more magnetic leaf springs being pressed; and
control the surgical tool in response thereto.

11. The apparatus according to claim 10, wherein the magnetic leaf springs are covered with a flexible sleeve.

12. The apparatus according to claim 10, wherein the input-receiving component is configured to receive an input via pressure that is applied to the magnetic leaf springs by a single finger or thumb of an operator.

13. The apparatus according to claim 10, wherein the input-receiving component is configured to receive an input via pressure that is applied to the magnetic leaf springs by two fingers.

14. The apparatus according to claim 10, wherein the magnetic leaf springs are distributed non-uniformly around a circumference of the handle.

15. The apparatus according to claim 10, wherein the apparatus is configured for performing an ophthalmic procedure on an eye of a patient using one or more surgical tools that have tips and wherein the robotic unit is configured to move the one or more surgical tools within the patient's eye.

16. The apparatus according to claim 15, wherein the computer processor is configured to:
determine movement of the location and orientation of the tip of the control-component tool based upon data received from the one or more location sensors; and
move the tip of the surgical tool within the patient's eye in a manner that corresponds with the movement of the location and orientation of the tip of the control-component tool.

17. The apparatus according to claim 10, wherein the input-receiving component is configured to be rotationally agnostic, such that magnetic flux that is generated by pressing the magnetic leaf springs is the same regardless of a roll orientation of the control-component tool relative to an operator's hand.

18. The apparatus according to claim 17, wherein the magnetic leaf springs are distributed uniformly around a circumference of handle.

19. An apparatus for performing a procedure on a patient using a surgical tool, the apparatus comprising:
a robotic unit configured to control the surgical tool; and
a control-component tool that is configured to be held by an operator, the control-component tool comprising:
a handle;
a first magnet disposed on a first side of a longitudinal axis of the handle and a second magnet disposed opposite the first magnet on a second side of the longitudinal axis of the handle, with a pole of each of the first and second magnets that is disposed closer to the longitudinal axis being the same as each other; and
a magnetometer that is disposed within the handle along a centerline of the first and second magnets along a radial direction of the handle but offset from centerlines of the first and second magnets along an axial direction of the handle;
a computer processor configured to:
receive a signal from the magnetometer that is indicative of magnetic flux that is generated by the magnetic structures being pressed; and
control the surgical tool in response thereto.

20. The apparatus according to claim 19, wherein the computer processor is configured to distinguish between the only the first magnet being pressed, only the second magnet being pressed, and both magnets being pressed based on the signal from the magnetometer.
